(19) United States
(12) Patent Application Publication
(10) Pub. No.: US 2007/0015719 A1
(43) Pub. Date: Jan. 18, 2007

(54) Nanoparticulate clarithromycin formulations

(75) Inventors: Scott Jenkins, Downingtown, PA (US); Gary G. Liversidge, West Chester, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/481,257

(22) Filed: Jul. 6, 2006

Related U.S. Application Data
(60) Provisional application No. 60/697,095, filed on Jul. 7, 2005.

(51) Int. Cl. A61K 31/7052

(52) U.S. Cl. 514/29; 977/906

(57) ABSTRACT

The present invention is directed to compositions comprising nanoparticulate macrolides such as clarithromycin, or a salt or derivative thereof, having improved bioavailability. The nanoparticulate macrolide particles of the composition have an effective average particle size of less than about 2000 nm and are useful in the treatment of infection and related diseases.

### CROSS-REFERENCE TO RELATED PATENT APPLICATIONS

This application claims the benefit under 35 U.S.C. § 119(e) to U.S. Provisional Application No. 60/697,095, filed on Jul. 6, 2005, which is incorporated herein in its entirety.

### FIELD

The invention relates generally to compounds and compositions useful in the treatment of infection and related diseases. More specifically, the invention relates to nanoparticulate macrolide compositions, such as clarithromycin compositions, having an effective average particle size of less than about 2000 nm. The invention also relates to methods of formulating and manufacturing nanoparticulate clarithromycin compositions, and to methods of treatment using the compositions.

### BACKGROUND OF THE INVENTION

The following discussion of the background of the invention is merely provided to aid the reader in understanding the invention and is not admitted to describe or constitute prior art to the invention.

An antibiotic is a therapeutic compound that slows or kills bacterial growth, but is generally harmless to the host. There are many classes of antibiotics, each with a slightly different utility, mode of action, or bacterial target. Exemplary antibiotic classes include: aminoglycosides, carbacephems, carbapenems, first, second, third and fourth generation cephalosporins, glycopeptides, macrolides, monobactams, penicillins, polypeptides, quinolones, sulfonamides, tetracyclines, and unclassified antibiotic compounds such as chloramphenicol, clindamycin, ethambutol, fosfomycin, furazolidone, isoniazid, linezolid, metroindazole, nitrofurantoin, pyrazinamide, quinupristin, dalfopristin, rifampin and spectinomycin.

One class of antibiotics, the macrolides, belong to the polyketide class of natural products. The macrolides are characterized by a macrocyclic ring, a large lactone ring to which one or more deoxy sugars, usually cladinose and desosamine, are attached. The lactone ring can be either 14, 15 or 16-membered. Macrolides function, generally, by inhibiting protein synthesis in bacteria via inhibition of 50S ribosome subunit formation. This inhibition of protein synthesis slows bacterial growth and division or kills the bacteria outright.

Some examples of macrolide antibiotics include: Azithromycin (CAS RN: 83905-01-5); Brefeldin A (CAS RN: 20350-15-6); Clarithromycin (CAS RN: 81103-11-9); Erythromycin (CAS RN: 114-07-8); Erythromycin Estolate (CAS RN: 3521-62-8); Erythromycin Ethyl Succinate (CAS RN: 1264-62-6); Erythromycin Stearate (CAS RN: 643-22-1); Josamycin (CAS RN: 16846-24-5); Kitasamycin (CAS RN: 1392-21-8); Lincomycin Hydrochloride (CAS RN: 859-18-7); Mepartricin (CAS RN: 11121-32-7); Midecamycin (CAS RN: 35457-80-8); Oleandomycin Phosphate (CAS RN: 7060-74-4); Oleandomycin Triacetate (CAS RN: 2751-09-9); Rokitamycin (CAS RN: 74014-51-0); Roxithromycin (CAS RN: 80214-83-1); Spiramycin (CAS RN: 8025-81-8); Tylosin (CAS RN: 74610-55-2); Tylosin Tartrate (CAS RN: 74610-55-2); Virginiamycin M (CAS RN: 21411-53-0).

A popular macrolide, erythromycin, is used as an antibiotic against many kinds of infections caused by gram-positive bacteria, including some beta-hemolytic streptococci, pneumococci and staphylococci as well as gram-negative bacteria and some fungi. It is also used also in the treatment of upper and lower respiratory tract infections caused by chlamydia trachomatis and intestinal amebiasis, and for the treatment of syphilis in patients who may be allergic to penicillin and the treating Legionnaire's disease.

A. Background Regarding Clarithromycin

Another macrolide, clarithromycin, has close structural and biological similarity to erythromycin. Clarithromycin, chemically known as 6-o-methyl erythromycin A, has a molecular weight of 747.85 and an empiric formula of C₃₈H₆₉NO₁₃.

Clarithromycin has the chemical structure of:

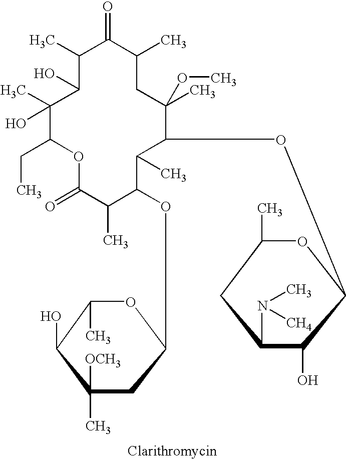

Clarithromycin is available under its generic name or several brand names, e.g., Biaxin® and Klacid®, from such companies as Abbott Laboratories (Biaxin®, Biaxin® XL), Andrx Pharmaceuticals, GenPharma, and Roxane Laboratories. Clarithromycin is commonly administered in tablets, extended-release tablets, or oral suspension.

Clarithromycin has been shown to be effective against a broad spectrum of gram-positive and gram-negative bacteria, and is used to treat both respiratory tract and soft tissue infections, and can be used to treat pharyngitis, tonsillitis, acute maxillary sinusitis, acute bacterial exacerbation of chronic bronchitis, pneumonia (especially atypical pneumonias associated with Chlamydia pneumoniae also known as TWAR), skin and skin structure infections, and, in HIV-infected and AIDS patients to prevent, and to treat, disseminated mycobacterium avium complex. Additionally, clarithromycin can be used to treat duodenal ulcers associated with Helicobacter pylori infections in combination with omeprazole.

Clarithromycin has similar antimicrobial spectrum as erythromycin, but is more effective against certain gram-negative bacteria, particularly Legionella pneumophilae. In addition to this bacteriostatic effect, clarithromycin also has bactericidal effect on certain strains such as Haemophilus influenzae, Streptococcus pneumoniae and Neisseria gonorrhoeae.

Clarithromycin compounds have been disclosed, for example, in U.S. Pat. No. 4,331,803 for “Novel Erythromycin Compounds;” U.S. Pat. No. 5,705,190 for “Controlled Release Formulation for Poorly Soluble Basic Drugs;” U.S. Pat. No. 5,786,338 for “Method of Treating Hypercholesterolemia with a Macrolide Antibiotic;” U.S. Pat. U.S. Pat. No. 5,844,105 for “Preparation of Crystal Form II of Clarithromycin;” U.S. Pat. No. 5,858,986 for “Crystal Form I of Clarithromycin;” U.S. Pat. No. 6,610,328 for “Amoxicillin-Clarithromycin Antibiotic Composition;” U.S. Pat. No. 6,642,276 for “Controlled Release Macrolide Pharmaceutical Formulations;” U.S. Pat. No. 6,987,175 for “Processes for Preparing Clarithromycin Polymorphs;” U.S. Pat. No. 6,812,216 for “11-C-Substituted Derivatives of Clarithromycin;” U.S. Pat. No. No. 6,809,188 for “Method of Preparing Clarithromycin;” U.S. Pat. No. 6,642,364 for “Process to Obtain Clarithromycin;” U.S. Pat. No. 6,624,292 for “Processes for Preparing Clarithromycin Polymorphs;” U.S. Pat. No. 6,617,436 for “Processes for Preparing Clarithromycin and Clarithromycin Intermediate, Essentially Oxime-Free Clarithromycin, and Pharmaceutical composition Comprising the Same;” U.S. Pat. No. No. 6,605,301 for “Dispersible Macrolide compounds and Method for Production Thereof;” U.S. Pat. No. 6,600,025 for “Intermediates, Process for Preparing Macrolide Antibiotic Agent Therefrom;” U.S. Pat. No. 6,599,886 for “Macrolide Intermediates in the Preparation of Clarithromycin;” U.S. Pat. No. 6,599,885 for “Derivatives of Erythromycin, Clarithromycin, Roxithromycin or Azithromycin with Antibiotic and Mucolytic Activity;” U.S. Pat. No. 6,599,884 for “Processes for Preparing Clarithromycin Polymorphs and Novel Polymorph IV;” U.S. Pat. No. 6,515,116 for “Method of Preparing Form II Crystals of Clarithromycin;” U.S. Pat. No. 6,506,886 for “Method of Preparing Form II Crystals of Clarithromycin;” U.S. Pat. No. 6,444,796 for “Method of Preparing Form II Crystals of Clarithromycin;” U.S. Pat. No. 6,297,015 for “Crohn's Disease Diagnostic and Treatment Methods and Compositions;” U.S. Pat. No. 6,174,865 for “Method of Treating Hypertriglyceridemia with an Erythromycin Compound;” U.S. Pat. No. 5,972,309 for “Identification of an Exogenous Intra-Erythrocytic Bacterium in patients Having Systemic Lupus Erythematosus, and Treatment;” U.S. Pat. No. 5,795,871 for “Pharmaceutical Composition for Treatment of Non-Small Cell Lung Cancer;” U.S. Pat. No. 5,795,563 for “Identification of an Exogenous Intra-Erythrocytic Bacterium in Patients Having Systemic Lupus Erythematosus, and Treatment;” U.S. Pat. No. 5,760,010 for Method of Treating Liver Disorders with a Macrolide Antibiotic;” U.S. Pat. No. 5,498,424 for “Method of Treating Obesity;” U.S. Pat. No. 5,945,405 for “Crystal Form 0 of Clarithromycin;” and U.S. Pat. No. 5,919,489 for “Process for Aqueous Granulation of Clarithromycin,” all of which are incorporated herein by reference.

Clarithromycin has high therapeutic value in the treatment of infection and related diseases. However, the bioavailability of clarithromycin remains limited. For example, clarithromycin has low aqueous solubility at physiological pH, and is also stable in acidic solutions; clarithromycin's absolute bioavailability following oral administration is 50%. Additionally, the rate and extent of absorption of conventional clarithromycin tablets is increased by food intake 30 minutes before dosing. The food requirement may prove burdensome and inconvenient for some patients, and treatment may be adversely affected by a lack of patient compliance. Accordingly, it would be desirable to formulate a more soluble—and more bioavailable—form of a macrolide, such as clarithromycin, and to eliminate the need to take the drug with food. The compounds and methods described herein satisfy these needs, as well as other problems associated with the administration of conventional macrolide drug formulations.

The present invention then, relates to nanoparticulate macrolide compositions, such as nanoparticulate clarithromycin compositions, or a salts or derivatives thereof, for the treatment of infection and related diseases.

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having associated with or adsorbed onto the surface thereof a non-crosslinked surface stabilizer. The '684 patent does not describe nanoparticulate compositions of macrolide antibiotics, such as clarithromycin.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using lodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate lododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” U.S. Pat. No. 6,582,285 for “Apparatus for Sanitary Wet Milling;” U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid Droplet Aerosols of Nanoparticulate Drugs;” U.S. Pat. No. 6,908,626 for “Compositions Having a Combination of Immediate Release and Controlled Release Characteristics;” U.S. Pat. No. 6,969,529 for “Nanoparticulate Compositions Comprising Copolymers of Vinyl Pyrrolidone and Vinyl Acetate as Surface Stabilizers;” U.S. Pat. No. 6,976,647 for “System and Method for Milling Materials;” U.S. Pat. No. 6,991,191 for “Method of Using a Small Scale Mill;” all of which are specifically incorporated by reference.

In addition, U.S. Patent Publication No.20020012675 Al, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate Compositions Having a Peptide as a Surface Stabilizer;” U.S. Patent Publication No. 20050233001 for “Nanoparticulate Megestrol Formulations;” U.S. Patent Publication No. 20050147664 for “Compositions Comprising Antibodies and Methods of Using the Same for Targeting Nanoparticulate Active Agent Delivery;” U.S. Patent Publication No. 20050063913 for “Novel Metaxalone Compositions;” U.S. Patent Publication No. 20050042177 for “Novel Compositions of Sildenafil Free Base;” U.S. Patent Publication No. 20050031691 for “Gel Stabilized Nanoparticulate Active Agent Compositions;” U.S. Patent Publication No. 20050019412 for “Novel Glipizide Compositions;” U.S. Patent Publication No. 20050004049 for “Novel Griseofulvin Compositions;” U.S. Patent Publication No. 20040258758 for “Nanoparticulate Topiramate Formulations;” U.S. Patent Publication No. 20040258757 for” Liquid Dosage Compositions of Stable Nanoparticulate Active Agents;” U.S. Patent Publication No. 20040229038 for “Nanoparticulate Meloxicam Formulations;” U.S. Patent Publication No. 20040208833 for “Novel Fluticasone Formulations;” U.S. Patent Publication No. 20040195413 for “Compositions and Method for Milling Materials;” U.S. Patent Publication No. 20040156895 for “Solid Dosage Forms Comprising Pullulan;” U.S. Patent Publication No. U.S. Patent Publication No. U.S. Patent Publication No. 20040156872 for “Novel Nimesulide Compositions;” U.S. Patent Publication No. 20040141925 for “Novel Triamcinolone Compositions;” U.S. Patent Publication No. 20040115134 for “Novel Nifedipine Compositions;” U.S. Patent Publication No. 20040105889 for “Low Viscosity Liquid Dosage Forms;” U.S. Patent Publication No. 20040105778 for “Gamma Irradiation of Solid Nanoparticulate Active Agents;” U.S. Patent Publication No. 20040101566 for “Novel Benzoyl peroxide compositions;” U.S. Patent Publication No. 20040057905 for “Nanoparticulate Beclomethasone Dipropionate Compositions;” U.S. Patent Publication No. 20040033267 for “Nanoparticulate Compositions of Angiogenesis Inhibitors;” U.S. Patent Publication No. 20040033202 for “Nanoparticulate Sterol Formulations and Novel Sterol Combinations;” U.S. Patent Publication No. 20040018242 for “Nanoparticulate Nystatin formulations;” U.S. Patent Publication No. 20040015134 for “Drug delivery Systems and Methods;” U.S. Patent Publication No. 20030232796 for “Nanoparticulate Polycosanol Formulations & Novel Polycosanol Combinations;” U.S. Patent Publication No. 20030215502 for “Fast Dissolving Dosage Forms Having Reduced Friability;” U.S. Patent Publication No. 20030185869 for “Nanoparticulate Compositions Having Lysozyme as a Surface Stabilizer;” U.S. Patent Publication No. 20030181411 for “Nanoparticulate Compositions of Mitogen-Activated Protein (MAP) Kinase Inhibitors;” U.S. Patent Publication No. 20030137067 for “Compositions Having a Combination of Immediate Release and Controlled Release Characteristics;” U.S. Patent Publication No. 20030108616 for “Nanoparticulate Compositions Comprising Copolymers of Vinyl Pyrrolidone and Vinyl Acetate as Surface Stabilizers;” U.S. Patent Publication No. 20030095928 for “Nanoparticulate Insulin;” U.S. Patent Publication No. 20030087308 for “Method for High Through- put Screening Using a Small Scale Mill or Microfluidics;” U.S. Patent Publication No. 20030023203 for “Drug Delivery Systems & Methods;” U.S. Patent Publication No. 20020179758 for “System and Method for Milling Materials;” and U.S. Patent Publication No. 20010053664 for “Apparatus for Sanitary Wet Milling,” describe nanoparticulate active agent compositions and are specifically incorporated by reference. None of these references describe compositions of nanoparticulate macrolides, such as clarithromycin.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” These patents are also hereby incorporated by reference.

While the high therapeutic value of the macrolide compounds such as clarithromycin are recognized in the art, poorly soluble compounds are limited in their bioavailability upon oral administration and can be difficult or impossible to formulate as safe and effective products for other types of administration. Thus, there is a need in the art for formulations comprising macrolides which have improved oral bioavailability and thus improved efficacy and/or may be suitable for other types of administration, such as parenteral administration. The present invention fills these needs.

The present invention then, relates to nanoparticulate compositions comprising macrolides, such as clarithromycin, which may be useful in the treatment and prevention of conditions and symptoms related to bacterial infections, or other diseases, disorders or conditions for which a macrolide would be therapeutic.

### SUMMARY

The present compositions and methods relate to nanoparticulate compositions comprising a macrolide, such as clarithromycin, or a salt or derivative thereof (referred to herein collectively as clarithromycin), and at least one surface stabilizer, wherein the nanoparticles of clarithromycin have an effective average particle size of less than about 2000 nm. In some embodiments, the surface stabilizer may be associated with the surface of the particles, for example, the surface stabilizer may be adsorbed onto the surface of the macrolide particle.

The compositions may include macrolide particles, such as clarithromycin particles, which are in a crystalline phase, an amorphous phase, a semi-crystalline phase, a semi-amorphous phase and mixtures thereof.

The compositions may include one or more surface stabilizers. For example, some compositions may include at least one primary and at least one secondary surface stabilizer. Exemplary surface stabilizers include, but are not limited to non-ionic surface stabilizers, ionic surface stabilizers, anionic surface stabilizers, cationic surface stabilizers, zwitterionic surface stabilizers and combinations thereof.

The invention also relates to nanoparticulate macrolides, such as clarithromycin or a salt or derivative thereof compositions, at least one surface stabilizer, and optionally one or more pharmaceutically acceptable excipients, carriers, and optionally one or more active agents useful for the treatment of infection and related conditions. By way of example, but not by way of limitation, such diseases, disorders, conditions and symptoms include infection by a broad spectrum of gram-positive and gram-negative bacteria; both respiratory tract and soft tissue infections; pharyngitis; tonsillitis; acute maxillary sinusitis; acute bacterial exacerbation of chronic bronchitis; pneumonia (especially atypical pneumonias associated with Chlamydia pneumoniae or TWAR); skin and skin structure infections; and, in HIV infected and AIDS patients, disseminated mycobacterium avium complex. Additionally, the compounds of the present invention may be used to treat duodenal ulcer associated with Helicobacter pylori infections in combination with omeprazole.

The nanoparticulate compositions may be formulated in any pharmaceutically acceptable formulation. By way of example, but not by way of limitation, pharmaceutically acceptable formulations may include: formulation for oral, pulmonary, intravenous, rectal, opthalmic, colonic, parenteral, intracisternal, intravaginal, intraperitoneal, local, buccal, nasal, and topical administration; dosage forms such as liquid dispersions, gels, aerosols, ointments, creams, tablets, sachets and capsules; dosage forms such as lyophilized formulations, fast melt formulations, controlled release formulations, delayed release formulations, extended release formulations, pulsatile release formulations, and mixed immediate release and controlled release formulations, or any combination of the above. In some embodiments, preferred formulations for administration may include oral tablets, capsules, sachets, solutions, dispersions and mixtures thereof.

The nanoparticulate macrolide compositions, such as clarithromycin, are proposed to exhibit improved pharmacokinetic profiles as compared to conventional macrolide compositions such as clarithromycin tablets. For example, the Cₘₐₓ and/or AUC of the nanoparticulate compositions may be greater than the Cₘₐₓ and/or AUC for conventional non-nanoparticulate compositions of the same macrolide administered at the same dosage while the Tₘₐₓ may be lower; any combination of an improved Cₘₐₓ, AUC and Tₘₐₓ profile may be exhibited by the nanoparticulate macrolide compositions as compared to conventional non-nanoparticulate compositions of the same macrolide. In further embodiments, the macrolide compositions may not produce significantly different absorption levels when administered under fed as compared to fasting conditions. In still other embodiments, the nanoparticulate compositions, when administered to a human in a fasted state is bioequivalent to administration of the composition to a subject in a fed state.

In some embodiments, the nanoparticulate macrolide compositions, such as nanoparticulate clarithromycin compositions, exhibit improved bioavailability as compared to conventional macrolide compositions. For example, upon administration to a mammal, the nanoparticulate macrolide compositions may redisperse such that the particles have an effective average particle size of less than about 2 microns.

The invention also relates to methods of making nanoparticulate compositions including macrolides, such as clarithromycin, or salt or derivative thereof. In some embodiments, the methods may include contacting particles of a macrolide with at least one surface stabilizer for a time and under conditions sufficient to provide a nanoparticulate macrolide composition having an effective average particle size of less than about 2000 nm. By way of example, but not by way of limitation, contacting may include milling, wet milling, homogenizing, precipitation, freezing, supercritical fluid particle generation techniques, emulsion techniques, or a combination thereof.

The invention also relates to methods of treatment using the nanoparticulate macrolide compositions, such as clarithromycin or a salt or derivative thereof. In some methods, a composition having a nanoparticulate clarithromycin or salt or derivative thereof, having an effective average particle size of less than about 2000 nm, and including at least one surface stabilizer, may be administered to a subject. In some methods, the composition may be administered orally, for example, as a tablet, in a therapeutically effective amount. By way of example, but not by way of limitation, the composition may be administered to treat diseases, disorders, symptoms or conditions that relate to bacterial infections. In other methods, the subject may be suffering from such a disease, disorder, symptom or condition. Additionally, other methods of treatment using the nanoparticulate compositions of the invention are known to those of skill in the art.

Both the foregoing summary of the invention and the following detailed description of the invention are exemplary and explanatory and are intended to provide further details of the invention as claimed. Other objects, advantages, and novel features will be readily apparent to those skilled in the art from the following detailed description of the invention.

### DESCRIPTION

A. Nanoparticulate Macrolide Compositions

The compositions and methods disclosed herein are directed to nanoparticulate compositions comprising a macrolide, such as clarithromycin, or a salt or derivative thereof (referred to herein collectively as clarithromycin), and preferably at least one surface stabilizer associated with or adsorbed on the surface of the drug particles. The clarithromycin particles are contemplated to have an effective average particle size of less than about 2000 nm.

Advantages of the nanoparticulate macrolide formulations, such as nanoparticulate clarithromycin formulations as compared to non-nanoparticulate compositions (e.g., microcrystalline or solubilized dosage forms) of the same macrolide, include but are not limited to: (1) smaller tablet or other solid dosage form size; (2) smaller doses of the drug required to obtain the same pharmacological effect; (3) improved pharmacokinetic profiles; (4) increased bioavailability; (5) substantially similar pharmacokinetic profiles of the nanoparticulate macrolide compositions when administered in the fed versus the fasted state; (6) bioequivalency of the nanoparticulate macrolide compositions when administered in the fed versus the fasted state; (7) an increased rate of dissolution; (8) an increased rate of absorption; and (9) the macrolide compositions can be used in conjunction with other active agents useful in the treatment of diseases, disorders, symptoms or conditions related to bacterial infections.

The present compositions and methods also relate to nanoparticulate macrolides, such as clarithromycin, or a salt or derivative thereof, compositions together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, bioadhesive or aerosol form, vaginal, nasal, rectal, ocular, otic, local (powders, ointments, or drops), buccal, intracistemal, intraperitoneal, or topical administrations, and the like.

In some embodiments, a preferred dosage form may be a solid dosage form, such as a tablet, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The present invention is described herein using several definitions, as set forth below and throughout the application.

The term “effective average particle size of less than about 2000 nm,” as used herein, means that at least about 50% of the nanoparticulate macrolide, such as clarithromycin particles have a size of less than about 2000 nm (by weight or by other suitable measurement technique, such as by number or by volume) when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable nanoparticulate macrolide, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the macrolide has not been subject to a heating step at or above the melting point of the macrolide in the preparation of the nanoparticles of the present invention.

The term “conventional” or “non-nanoparticulate” active agent shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixtures thereof irrespective of their size, shape or morphology.

B. Characteristics of the Nanoparticulate Macrolide Compositions

1. Increased Bioavailability

The nanoparticulate macrolide, such as clarithromycin, formulations of the invention are contemplated to exhibit increased bioavailability as compared to non-nanoparticulate formulations of the same macrolide. Moreover, the nanoparticulate compositions are expected to require smaller doses, and smaller tablet or other solid dosage form size as compared to prior conventional non-nanoparticulate formulations of the same macrolide.

The increased bioavailability of the nanoparticulate formulations is also likely to result in a dosage form that exhibits greater drug absorption than conventional formulations of the same macrolide.

2. Improved Pharmacokinetic Profiles

The nanoparticulate macrolide compositions, such as clarithromycin, described herein may also exhibit desirable pharmacokinetic profiles when administered to mammalian subjects. Exemplary desirable pharmacokinetic profiles of the nanoparticulate compositions preferably include, but are not limited to: (1) a Cₘₐₓ for a macrolide such as clarithromycin, or a derivative or salt thereof, when assayed in the plasma of a mammalian subject following administration, that is preferably greater than the Cₘₐₓ for a non-nanoparticulate formulation of the same macrolide administered at the same dosage; and/or (2) an AUC for a macrolide such as clarithromycin or a derivative or a salt thereof, when assayed in the plasma of a mammalian subject following administration, that is preferably greater than the AUC for a non-nanoparticulate formulation of the same macrolide, administered at the same dosage; and/or (3) a Tₘₐₓ for a macrolide such as clarithromycin or a derivative or a salt thereof, when assayed in the plasma of a mammalian subject following administration, that is preferably less than the Tₘₐₓ for a non-nanoparticulate formulation of the same macrolide, administered at the same dosage. The desirable pharmacokinetic profile, as used herein, is the pharmacokinetic profile measured after the initial dose of the macrolide such as clarithromycin or derivative or a salt thereof.

In one embodiment, a composition comprising at least one nanoparticulate macrolide, such as clarithromycin or a derivative or salt thereof exhibits in comparative pharmacokinetic testing with a non-nanoparticulate formulation of the same clarithromycin (e.g., BIAXIN® or KLACID®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate clarithromycin formulation.

In another embodiment, the composition comprising at least one nanoparticulate clarithromycin or a derivative or salt thereof, exhibits in comparative pharmacokinetic testing with a non-nanoparticulate formulation of the same clarithromycin (e.g., BIAXIN® or KLACID®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate clarithromycin formulation.

In yet another embodiment, the composition comprising at least one nanoparticulate clarithromycin or a derivative or salt thereof, exhibits in comparative pharmacokinetic testing with a non-nanoparticulate formulation of the same clarithromycin (e.g., BIAXIN® or KLACID®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate clarithromycin formulation.

The compositions can be formulated in any way as described herein and as known to those of skill in the art.

3. The Pharmacokinetic Profiles of the Macrolide Compositions are not Affected by the Fed or Fasted State of the Subject Ingesting the Compositions

In some embodiments, the pharmacokinetic profiles of the nanoparticulate macrolide, such as clarithromycin, compositions are not substantially affected by the fed or fasted state of a subject ingesting the composition. This means that there would be little or no appreciable difference in the quantity of drug absorbed or the rate of drug absorption when the nanoparticulate macrolide, such as clarithromycin, compositions are administered in the fed or fasted state.

For conventional clarithromycin formulations, i.e., BIAXIN or KLACID, the absorption of clarithromycin is increased when administered with food. This difference in absorption observed with conventional clarithromycin formulations is undesirable. The nanoparticulate clarithromycin formulations of the invention are proposed to overcome this problem, as the clarithromycin formulations are likely to reduce or preferably substantially eliminate significantly different absorption levels when administered under fed as compared to fasting conditions.

Benefits of a dosage form which substantially eliminates the effect of food include an increase in subject convenience, thereby increasing subject compliance, as the subject does not need to ensure that they are taking a dose either with or without food. This can be significant, as with poor subject compliance an increase in the medical condition for which the drug is being prescribed may be observed.

4. Bioequivalency of Macrolide Compositions When Administered in the Fed Versus the Fasted State

In one embodiment, administration of a nanoparticulate macrolide, such as clarithromycin, composition to a subject in a fasted state is bioequivalent to administration of the composition to a subject in a fed state. The difference in absorption of the nanoparticulate macrolide compositions, when administered in the fed versus the fasted state, preferably is less than about 100%, less than about 90%, less than about 80%, less than about 70%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In some embodiments, the invention encompasses compositions comprising at least one nanoparticulate macrolide (e.g., clarithromycin) wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Macrolide Compositions of the Invention

The nanoparticulate macrolide compositions, such as nanoparticulate clarithromycin compositions, are proposed to have unexpectedly dramatic dissolution profiles. Rapid dissolution of an administered active agent is preferable, as faster dissolution generally leads to faster onset of action and greater bioavailability. To improve the dissolution profile and bioavailability of the macrolide it would be useful to increase the drug's dissolution so that it could attain a level close to 100%.

In some embodiments, the macrolide compositions (e.g., clarithromycin) have a dissolution profile in which within about 5 minutes at least about 20% of the composition is dissolved. In other embodiments, at least about 30% or about 40% of the macrolide composition is dissolved within about 5 minutes. In yet other embodiments, preferably at least 40%, about 50%, about 60%, about 70%, or about 80% of the macrolide composition is dissolved within about 10 minutes. In further embodiments, at least about 70%, about 80%, about 90%, or about 100% of the macrolide composition is dissolved within 20 minutes.

Dissolution may be measured in a medium which is discriminating. A discriminating dissolution medium is one that will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersability of the Macrolide Compositions

An additional feature of the macrolide, such as clarithromycin, compositions is that the compositions redisperse such that the effective average particle size of the redispersed clarithromycin particles is less than about 2 microns. If upon administration, the nanoparticulate macrolide compositions did not redisperse to a substantially nanoparticulate size, then the dosage form may lose the benefits afforded by formulating the macrolide into a nanoparticulate size.

Not wishing to be bound by any theory, it is proposed that the nanoparticulate active agent compositions benefit from the small particle size of the active agent; if the active agent does not disperse into the small particle sizes upon administration, them “clumps” or agglomerated active agent particles are formed, owing to the extremely high surface free energy of the nanoparticulate system and the thermodynamic driving force to achieve an overall reduction in free energy. With the formulation of such agglomerated particles, the bioavailability of the dosage form my fall.

Moreover, the nanoparticulate macrolide, such as clarithromycin, compositions of the invention are proposed to exhibit dramatic redispersion of the nanoparticulate macrolide particles upon administration to a mammal, such as a human or animal, as demonstrated by reconstitution/redispersion in a biorelevant aqueous media such that the effective average particle size of the redispersed macrolide particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, water, aqueous electrolyte solutions or aqueous solutions of any salt, acid, or base, or a combination thereof, which exhibit the desired pH and ionic strength. Such redispersion in a biorelevant media is predictive of in vivo efficacy of the macrolide dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments, the nanoparticulate macrolide compositions of the invention redisperse upon administration to a mammal, upon introduction to any suitable media, including a biorelevant media, to an effective average particle size selected from the group consisting of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example section of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Macrolide Compositions Used in Conjunction with Other Active Agents

The nanoparticulate macrolide, such as clarithromycin, compositions can additionally comprise one or more compounds useful in the treatment of infection and related diseases, or the clarithromycin compositions can be administered in conjunction with such a compound. Examples of such compounds include but are not limited to antibiotics, anti-virals (e.g., azidothymidine (“AZT”), didanosine (“DDI”), tenofovir (“TDF”), amdoxovir (“DAPD”), lamivudine (“3TC”), emtricitabine (“FTC”), zalcitabine (“DOC”), saquinavir, nelfinavir, aprenavir, non-nucleoside reverse transcriptase inhibitors, multi-drug resistance), anti-fungals (e.g., allylamines, antimetabolites, azoles such as miconazole and clotrimazole, chitin synthase inhibitors, glucan synthesis inhibitors, polyenes,), anti-inflammatories (e.g., diclofenac diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic acid tolmetinpain) pain relievers, fever reducers, muscle-relaxants, esophageal and stomach acid relievers such as omeprazole and the like.

C. Nanoparticulate Macrolide Compositions

The compositions and methods described herein relate to compositions comprising macrolides such clarithromycin, or a salt or derivative thereof, and at least one surface stabilizer. The surface stabilizers preferably are adsorbed on, or associated with, the surface of the macrolide particles. In some embodiments, the surface stabilizers preferably physically adhere on, or associate with, the surface of the nanoparticulate macrolide particles, but do not chemically react with the macrolide particles or itself. In some embodiments, individually adsorbed molecules of the surface stabilizer are essentially free of intermolecular cross-linkages.

The present compositions also relate to macrolide compositions together with one or more non-toxic physiologically acceptable carriers, adjuvants, or vehicles, collectively referred to as carriers. The compositions may be formulated for oral administration in solid, liquid, or aerosol form, for parenteral injection (e.g., intravenous, intramuscular, or subcutaneous), as a bioadhesive, a vaginal, nasal, rectal, ocular, local (powders, ointments or drops), buccal, intracisternal, intraperitoneal, or topical administration, and the like.

1. Macrolide Particles

The compositions of the invention comprise particles of macrolides such as clarithromycin, or a salt of derivative thereof. The particles may be in a crystalline phase, semi-crystalline phase, amorphous phase, semi-amorphous phase, or a combination thereof.

2. Surface Stabilizers

The choice of a surface stabilizer for macrolides, such as clarithromycin, is non-trivial and required extensive experimentation to realize a desirable formulation. Accordingly, the present invention is directed to the surprising discovery that nanoparticulate macrolide compositions can be made.

Combinations of more than one surface stabilizers may be used in the compositions and methods. Useful surface stabilizers which can be employed include, but are not limited to, known organic and inorganic pharmaceutical excipients. Such excipients include various polymers, low molecular weight oligomers, natural products, and surfactants. Exemplary surface stabilizers include nonionic, anionic, cationic, ionic, and zwitterionic surfactants or compounds.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Speciality Chemicals)); polyethylene glycols (e.g., Carbowaxs® 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic® 908, also known as Poloxamine™ 908, which is a tetrafunctional block copolymer derived from sequential addition of propylene oxide and ethylene oxide to ethylenediamine (BASF Wyandotte Corporation, Parsippany, N.J.); Tetronic® 1508 (T-1508) (BASF Wyandotte Corporation), Tritons® X-200, which is an alkyl aryl polyether sulfonate (Rohm and Haas); Crodestas™ F-110, which is a mixture of sucrose stearate and sucrose distearate (Croda Inc.); p-isononylphenoxypoly-(glycidol), also known as Olin®-1OG or Surfactant™ 10-G (Olin Chemicals, Stamford, Conn.); Crodestas™ SL-40 (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUA™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[dially dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄is C₆H₅CH₂, and one of R₁-R₄ comprises a least one halogen;

(x) two of R₁-R₄ are CH₃, one of R₁-R₄is C₆H₅CH₂, and one of R₁-R₄ comprises a least one cyclic fragment;

(xi) two of R₁-R₄ are CH₃ and one of R₁-R₄is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCI, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In some embodiments, one or more surface stabilizers may include copovidone (e.g., Plasdone S630, which comprises random copolymers of vinyl acetate and vinyl pyrrolodine) and docusate sodium.

Many of the surface stabilizers are known pharmaceutical excipients and are commercially available and/or can be prepared by techniques known in the art. See e.g., Handbook of Pharmaceutical Excipients, published jointly by the American Pharmaceutical Association and The Pharmaceutical Society of Great Britain (The Pharmaceutical Press, 2000), specifically incorporated by reference, describing many known pharmaceutical excipients in detail.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, include but are not limited to colloidal silicon dioxide, such as Aerosil® 200, talc, stearic acid, magnesium stearate, calcium stearate, and silica gel.

Examples of sweeteners may include any natural or artificial sweetener, such as sucrose, xylitol, sodium saccharin, cyclamate, aspartame, and acsulfame. Examples of flavoring agents may include Magnasweet® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Examples of buffers include phosphate buffer, citrate buffers and buffers made from other organic acids.

Examples of wetting or dispersing agents include a naturally-occurring phosphatide, for example, lecithin or condensation products of n-alkylene oxide with fatty acids, for example, polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyethylene sorbitan monooleate.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Aqueous suspensions comprising the nanoparticulate macrolide may be in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth and gum acadia.

4. Nanoparticulate Macrolide Particle Size

The nanoparticulate macrolide such as clarithromycin compositions are proposed to include nanoparticulate macrolide, such as clarithromycin, or a salt or derivative thereof, particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the macrolide particles have a particle size of less than the effective average, by weight (or by other suitable measurement technique, such as by volume, number, etc.), i.e., less than about 2000 nm, 1900 nm, 1800 nm, etc., when measured by the above-noted techniques. Preferably, at least about 60%, at least about 70%, at least about 90%, or at least about 95% of the clarithromycin particles have a particle size of less than the effective average, i.e., less than about 2000 nm, 1900 nm, 1800 nm, 1700 nm, etc.

In the present invention, the value for D50 of a nanoparticulate macrolide composition is the particle size below which 50% of the macrolide particles fall, by weight (or by other suitable measurement technique, such as by volume, number, etc.). Similarly, D90 is the particle size below which 90% of the macrolide particles fall, by weight (or by other suitable measurement technique, such as by volume, number, etc.).

5. Concentration of Macrolides and Surface Stabilizers

The relative amounts of macrolides, such as clarithromycin, or a salt or derivative thereof, and one or more surface stabilizers may vary. In some embodiments, the optimal amount of the individual components may depend, for example, upon the particular macrolide selected, the hydrophilic lipophilic balance (HLB), melting point, and the surface tension of water solutions of the stabilizer, etc.

For example, in some embodiments, the concentration of a macrolide, such as clarithromycin may vary from about 99.5% to about 0.001%, from about 95% to about 0.1%, or from about 90% to about 0.5%, by weight, based on the total combined dry weight of the clarithromycin and at least one surface stabilizer, not including other excipients.

In other embodiments, the concentration of the at least one surface stabilizer may vary from about 0.01% to about 99.5% by weight, from about 0.1% to about 95% by weight, from about 0.5% to about 90% by weight, from about 5.0% to about 99.9% by weight, and from about 10% to about 99.5% by weight, based on the total combined dry weight of clarithromycin and at least one surface stabilizer, not including other excipients. Any combination of the above weight % ratios is also contemplated.

6. Exemplary Nanoparticulate Clarithromycin Tablet Formulations

Several exemplary clarithromycin tablet formulations are provided below. These examples are not intended to limit the claims in any respect, but rather to provide exemplary tablet formulations of clarithromycin which can be utilized in the methods of the invention. Such exemplary tablets can also comprise a coating agent.

Exemplary Nanoparticulate
Clarithromycin Tablet Formulation #1

| Component | g/Kg |
|---|---|
| Clarithromycin | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate
Clarithromycin Tablet Formulation #2

| Component | g/Kg |
|---|---|
| Clarithromycin | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate
Clarithromycin Tablet Formulation #3

| Component | g/Kg |
|---|---|
| Clarithromycin | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate
Clarithromycin Tablet Formulation #4

| Component | g/Kg |
|---|---|
| Clarithromycin | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

D. Methods of Making Nanoparticulate Macrolide Compositions

The nanoparticulate macrolide such as clarithromycin, or a salt or derivative thereof, compositions can be made using, for example, milling, homogenization, precipitation, freezing, supercritical particle generation, or template emulsion techniques. Exemplary methods of making nanoparticulate compositions are described in the '684 patent. Methods of making nanoparticulate compositions are also described in U.S. Pat. No. U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate macrolide compositions or dispersions can be utilized in solid or liquid dosage formulations, such as liquid dispersions, gels, aerosols, ointments, creams, bioadhesives, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Macrolide Dispersions

Milling a macrolide, such as clarithromycin, or a salt or derivative thereof, to obtain a nanoparticulate dispersion comprises dispersing the macrolide particles in a liquid dispersion medium in which the macrolide is poorly soluble, followed by applying mechanical means in the presence of grinding media to reduce the particle size of the macrolide to the desired effective average particle size. The dispersion medium can be, for example, water, safflower oil, ethanol, t-butanol, glycerin, polyethylene glycol (PEG), hexane, or glycol. In some embodiments, a preferred dispersion medium is water.

The macrolide particles can be reduced in size in the presence of at least one surface stabilizer. Alternatively, macrolide particles can be contacted with one or more surface stabilizers after attrition. Other compounds, such as a diluent, can be added to the macrolide/surface stabilizer composition during the size reduction process. Dispersions can be manufactured continuously or in a batch mode.

2. Precipitation to Obtain Nanoparticulate Macrolide Compositions

Another method of forming the desired nanoparticulate macrolide such as clarithromycin, or a salt or derivative thereof, compositions is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the macrolide in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Macrolide Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a macrolide such as clarithromycin, or a salt or derivative thereof, in a liquid dispersion medium, followed by subjecting the dispersion to homogenization to reduce the particle size of the macrolide to the desired effective average particle size. The macrolide particles can be reduced in size in the presence of at least one surface stabilizer. Alternatively, the macrolide particles can be contacted with one or more surface stabilizers either before or after attrition. Other compounds, such as a diluent, can be added to the macrolide/surface stabilizer composition either before, during, or after the size reduction process. Dispersions can be manufactured continuously or in a batch mode.

4. Cryogenic Methodologies to Obtain Nanoparticulate Macrolide Compositions

Another method of forming the desired nanoparticulate macrolide such as clarithromycin, or a salt or derivative thereof, composition is by spray freezing into liquid (SFL). This technology comprises an organic or organoaqueous solution of macrolide with stabilizers, which is injected into a cryogenic liquid, such as liquid nitrogen. The droplets of the macrolide solution freeze at a rate sufficient to minimize crystallization and particle growth, thus formulating nanostructured macrolide particles. Depending on the choice of solvent system and processing conditions, the nanoparticulate macrolide particles can have varying particle morphology. In the isolation step, the nitrogen and solvent are removed under conditions that avoid agglomeration or ripening of the macrolide particles.

As a complementary technology to SFL, ultra rapid freezing (URF) may also be used to created equivalent nanostructured macrolide particles with greatly enhanced surface area. URF comprises an organic or organoaqueous solution of macrolide with stabilizers onto a cryogenic substrate.

5. Emulsion Methodologies to Obtain Nanoparticulate Clarithromycin Compositions

Another method of forming the desired nanoparticulate macrolide such as clarithromycin, or a salt or derivative thereof, compositions is by template emulsion. Template emulsion creates nanostructured macrolide particles with controlled particle size distribution and rapid dissolution performance. The method comprises an oil-in-water emulsion that is prepared, then swelled with a non-aqueous solution comprising the macrolide and stabilizers. The particle size distribution of the macrolide particles is a direct result of the size of the emulsion droplets prior to loading with the macrolide, a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured macrolide particles are recovered. Various macrolide particle morphologies can be achieved by appropriate control of processing conditions.

E. Methods of Using the Nanoparticulate Macrolide Compositions of the Invention

The invention provides a method of increasing bioavailability (e.g., increasing the plasma levels) of a macrolide such as clarithromycin, or a salt or derivative thereof, in a subject. Such a method comprises orally administering to a subject an effective amount of a composition comprising an clarithromycin.

In one embodiment of the invention, the nanoparticulate clarithromycin composition, in accordance with standard pharmacokinetic practice, has a bioavailability that is about 50% greater, about 40% greater, about 30% greater, about 20% greater, or about 10% greater than a conventional dosage form.

Additionally, in another embodiment of the invention, the compositions when tested in fasting subjects in accordance with standard pharmacokinetic practice, are proposed to produce a maximum blood plasma concentration profile in less than about 6 hours, less than about 5 hours, less than about 4 hours, less than about 3 hours, less than about 3 hours, less than about 1 hour or less than about 30 minutes after the initial dose of the composition.

The compositions of the invention are useful in the treatment of diseases, disorders, conditions and symptoms related to infection. By way of example, but not by way of limitation, such diseases, disorders, conditions and symptoms include infection by a broad spectrum of gram-positive and gram-negative bacteria; both respiratory tract and soft tissue infections; pharyngitis; tonsillitis; acute maxillary sinusitis; acute bacterial exacerbation of chronic bronchitis; pneumonia (especially atypical pneumonias associated with Chlamydia pneumoniae or TWAR); skin and skin structure infections; and, in HIV and AIDS patients, disseminated mycobacterium avium complex. Additionally, the compounds of the present invention may be used to treat duodenal ulcer associated with Helicobacter pylori infections in combination with omeprazole.

The macrolide such as clarithromycin, or a salt or derivative thereof compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), as a bioadhesive, or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate macrolide such as clarithromycin, or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a macrolide such as clarithromycin, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

‘Therapeutically effective amount’ as used herein with respect to, for example, a clarithromycin dosage shall mean that dosage that provides the specific pharmacological response for which a clarithromycin is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that macrolide dosages are, in particular instances, measured as oral dosages, or with reference to drug levels as measured in blood.

One of ordinary skill will appreciate that effective amounts of a macrolide such as clarithromycin can be determined empirically and can be employed in pure form or, where such forms exist, in pharmaceutically acceptable salt, ester, or prodrug form. Actual dosage levels of a macrolide such as clarithromycin in the nanoparticulate compositions of the invention may be varied to obtain an amount of the macrolide that is effective to obtain a desired therapeutic response for a particular composition and method of administration. The selected dosage level therefore depends upon the desired therapeutic effect, the route of administration, the potency of the administered macrolide, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### F. EXAMPLES

The following example is provided to illustrate the present invention. It should be understood, however, that the invention is not to be limited to the specific conditions or details described in the example. Throughout the specification, any and all references to a publicly available document, including a U.S. patent, are specifically incorporated by reference.

### Example 1.

The purpose of this example is to prepare a composition comprising a nanoparticulate clarithromycin or a salt or a derivative thereof.

An aqueous dispersion of 5% (w/w) clarithromycin, combined with one or more surface stabilizers, such as hydroxypropyl cellulose (HPC-SL) and dioctylsulfosuccinate (DOSS), could be milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical Co.) (e.g., at an 89% media load). In an exemplary process, the mixture could be milled at a speed of 2500 rpm for 60 minutes.

Following milling, the particle size of the milled clarithromycin particles can be measured, in deionized distilled water, using a Horiba LA 910 particle size analyzer. For a successful composition, the initial mean and/or D50 milled clarithromycin particle size is expected to be less than 2000 nm.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate clarithromycin, or a salt or derivative thereof, composition comprising:
(a) particles of clarithromycin, or a salt or derivative thereof, having an effective average particle size of less than about 2000 nm; and
(b) at least one surface stabilizer.

2. The composition of claim 1, wherein the clarithromycin is in a crystalline phase, an amorphous phase, a semi-crystalline phase, a semi amorphous phase, or mixtures thereof.

3. The composition of claim 1, wherein the effective average particle size of the particles of clarithromycin or a salt or derivative thereof is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the nanoparticulate clarithromycin has improved bioavailability as compared to conventional clarithromycin tablets.

5. The composition of claim 1, wherein the composition is formulated:
(a) for administration selected from the group consisting of oral, pulmonary, intravenous, rectal, opthalmic, colonic, parenteral, intracisternal, intravaginal, intraperitoneal, local, buccal, nasal, and topical administration;
(b) into a dosage form selected from the group consisting of liquid dispersions, gels, aerosols, ointments, creams, tablets, sachets and capsules;
(c) into a dosage form selected from the group consisting of lyophilized formulations, fast melt formulations, controlled release formulations, delayed release formulations, extended release formulations, pulsatile release formulations, and mixed immediate release and controlled release formulations; or
(d) any combination of (a), (b), and (c).

6. The composition of claim 1, wherein the composition is formulated for administration selected from the group consisting of oral tablets, capsules, sachets, solutions, dispersions and mixtures thereof.

7. The composition of claim 1, wherein the composition further comprises one or more pharmaceutically acceptable excipients, carriers, or a combination thereof.

8. The composition of claim 1, wherein:
(a) the amount of clarithromycin is selected from the group consisting of from about 99.5% to about 0.001%, from about 95% to about 0.1%, and from about 90% to about 0.5%, by weight, based on the total combined weight of clarithromycin and at least one surface stabilizer, not including other excipients;
(b) at least one surface stabilizer is present in an amount selected from the group consisting of from about 0.01% to about 99.5% by weight, from about 0.1% to about 95% by weight, from about 0.5% to about 90% by weight, from about 5.0% to about 99.9% by weight, and from about 10% to about 99.5% by weight, based on the total combined dry weight of clarithromycin and at least one surface stabilizer, not including other excipients; or
(c) a combination of (a) and (b).

9. The composition of claim 1, comprising at least one primary surface stabilizer and at least one secondary surface stabilizer.

10. The composition of claim 1, wherein at least one surface stabilizer is selected from the group consisting of a non-ionic surface stabilizer, an anionic surface stabilizer, a cationic surface stabilizer, a zwitterionic surface stabilizer, and an ionic surface stabilizer.

11. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂ p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone; a cationic polymer, a cationic biopolymer, a cationic polysaccharide, a cationic cellulosic, a cationic alginate, a cationic nonpolymeric compound, cationic phospholipids; cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

12. The composition of claim 1, additionally comprising one or more active agents useful for the treatment of infection and related conditions.

13. The composition of claim 1 wherein:
(a) the particles of clarithromycin or a salt or derivative thereof redisperse such that the particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm;
(b) the composition redisperses in a biorelevant medium such that the clarithromycin particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm; or
(c) a combination of (a) and (b).

14. The composition of claim 13, wherein the biorelevant medium is selected from the group consisting of water, aqueous electrolyte solutions, aqueous solutions of a salt, aqueous solutions of an acid, aqueous solutions of a base, and combinations thereof.

15. The composition of claim 1 wherein:
(a) the Tₘₐₓ of the nanoparticulate clarithromycin composition, when assayed in the plasma of a mammalian subject following administration, is less than the Tₘₐₓ for a non-nanoparticulate composition of the same clarithromycin, administered at the same dosage;
(b) the Cₘₐₓ of the nanoparticulate clarithromycin composition, when assayed in the plasma of a mammalian subject following administration, is greater than the Cₘₐₓ for a non-nanoparticulate composition of the same clarithromycin, administered at the same dosage;
(c) the AUC of the nanoparticulate clarithromycin composition, when assayed in the plasma of a mammalian subject following administration, is greater than the AUC for a non-nanoparticulate composition of the same clarithromycin, administered at the same dosage; or
(d) any combination thereof.

16. The composition of claim 1, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same clarithromycin, administered at the same dosage;
(b) the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same clarithromycin, administered at the same dosage;
(c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the same clarithromycin, administered at the same dosage; or
(d) any combination of (a), (b), and (c).

17. The composition of claim 1 which does not produce significantly different absorption levels when administered under fed as compared to fasting conditions.

18. The composition of claim 17, wherein the difference in absorption of the active agent composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

19. The composition of claim 1, wherein administration of the composition to a human in a fasted state is bioequivalent to administration of the composition to a subject in a fed state.

20. The composition of claim 19, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

21. A method of making a nanoparticulate clarithromycin, or a salt or derivative thereof, composition comprising: contacting particles of clarithromycin with at least one surface stabilizer for a time and under conditions sufficient to provide a nanoparticulate clarithromycin composition having an effective average particle size of less than about 2000 nm.

22. The method of claim 21, wherein contacting comprises milling, wet milling, homogenizing, precipitation, freezing, supercritical fluid particle generation techniques, emulsion techniques or a combination thereof.

23. A method for treating a subject in need, wherein the subject is suffering from an infection or related condition, comprising: administering a therapeutically effective amount of a composition comprising:
(a) particles of clarithromycin, or a salt or derivative thereof, having an effective average particle size of less than about 2000 nm; and
(b) at least one surface stabilizer.
